(19) United States
(12) Patent Application Publication
(10) Pub. No.: US 2021/0123901 A1
(43) Pub. Date: Apr. 29, 2021

(54) QUALITY INSPECTION METHOD FOR POLYURETHANE SAMPLES, ELECTRONIC NOSE DEVICE AND STORAGE MEDIUM

(71) Applicant: COVESTRO INTELLECTUAL PROPERTY GMBH & CO. KG, Leverkusen (DE)

(72) Inventors: Erika ZHU, Shanghai (CN); Irene LI, Shanghai (CN)

(73) Assignee: Covestro Intellectual Property GmbH & Co. KG, Leverkusen (DE)

(21) Appl. No.: 17/041,485

(22) PCT Filed: Mar. 26, 2019
(86) PCT No.: PCT/EP2019/057594

§ 371 Date: Sep. 25, 2020

(30) Foreign Application Priority Data
Mar. 28, 2018 (CN) 201810262856.4

(51) Int. Cl. G01N 33/44; G01N 33/00

(52) U.S. Cl. CPC G01N 33/442; G01N 33/0047; G01N 33/0001; G01N 33/0034

(57) ABSTRACT

The present invention relates to a quality inspection method for polyurethane samples, an electronic nose device and a storage medium. The quality inspection method for a polyurethane sample comprises the following steps: comparing measured spectrum information of a polyurethane sample to be inspected to reference spectrum information of a plurality of inspected polyurethane samples to determine the reference spectrum information with a predetermined confidence interval to which the measured spectrum information conforms, wherein each of the reference spectrum information is associated with property information of corresponding inspected polyurethane samples; and obtaining a quality inspection result of the polyurethane sample to be inspected on the basis of the property information associated with the determined reference spectrum information.

### TECHNICAL FIELD

The present invention relates to the field of detection of polyurethane materials, in particular to a quality inspection method for polyurethane samples, an electronic nose device and a storage medium.

### BACKGROUND OF THE INVENTION

Polyurethane materials have been widely used in fields like automobile and furniture, and are made into various automobile interiors, seats, dashboards, mattresses, sofa, and so on. However, the VOC (volatile organic compounds) and smells of the polyurethane materials can cause inconvenience and even health issues, so that people want to buy polyurethane products having less VOC and slight smell. Thus, it becomes very important to objectively detect the VOC and smell of the polyurethane products.

However, the detection, especially of the smell, is currently performed mainly by smelling by assessors or other experienced experts, and the problem thereof is that owing to the subjectivity of smell test, different assessors/experts might give different smell scores to the same product, besides, it is also impossible to assign one or several assessors/experts to test the smell of each batch of products during the actual production process of the polyurethane material. Thus, how to inspect the quality of polyurethane materials quickly, objectively and accurately has become a pressing concern in the industry.

### SUMMARY OF THE INVENTION

The present invention is proposed to overcome one or more of the above-mentioned defects or other defects, and the technical solutions adopted are described below.

According to a first aspect of the present invention, a quality inspection method for a polyurethane sample is provided, which includes the following steps: comparing measured spectrum information of a polyurethane sample to be inspected to reference spectrum information of a plurality of inspected polyurethane samples to determine the reference spectrum information with a predetermined confidence interval to which the measured spectrum information conforms, wherein each of the reference spectrum information is associated with property information of corresponding inspected polyurethane samples, and obtaining a quality inspection result of the polyurethane sample to be inspected on the basis of the property information associated with the determined reference spectrum information.

Further, in the quality inspection method for a polyurethane sample according to the first aspect of the present invention, the property information may include smell evaluation information and/or a mark indicating whether the sample is a qualified sample.

Further, the quality inspection method for a polyurethane sample according to the first aspect of the present invention may also comprise the step of measuring the polyurethane sample to be inspected to obtain the measured spectrum information.

Further, the quality inspection method for a polyurethane sample according to the first aspect of the present invention may also comprise the steps of selecting at least one piece of reference spectrum information from the reference spectrum information of the plurality of inspected polyurethane samples; comparing the selected at least one piece of reference spectrum information to the measured spectrum information; and analyzing the main chemical substances that cause spectrum information differences.

Further, the quality inspection method for a polyurethane sample according to the first aspect of the present invention may also comprise the steps of selecting at least one piece of reference spectrum information from the reference spectrum information of the plurality of inspected polyurethane samples; comparing the selected at least one piece of reference spectrum information to the measured spectrum information for one or more substances comprised in the polyurethane sample to be inspected, in particular constituting the sample to be inspected; and determining a content difference of the one or more substances in at least one inspected polyurethane sample corresponding to the at least one reference spectrum information and in the polyurethane sample to be inspected corresponding to the measured spectrum information.

Further, the quality inspection method for a polyurethane sample according to the first aspect of the present invention also comprises the step of detecting release of the one or more chemical substances constituting the polyurethane sample to be inspected in the air.

Further, in the quality inspection method for a polyurethane sample according to the first aspect of the present invention, the smell evaluation information may be information based on VDA270 criterion.

According to a second aspect of the present invention, an electronic nose device is provided, which comprises:

a database unit storing databases of reference spectrum information of a plurality of inspected polyurethane samples and property information of inspected polyurethane samples associated with each of the reference spectrum information;

a confidence interval comparing unit configured to compare the measured spectrum information of the polyurethane sample to be inspected to the reference spectrum information of the plurality of inspected polyurethane samples to determine the reference spectrum information with a predetermined confidence interval to which the measured spectrum information conforms; and

a quality inspection result outputting unit configured to obtain a quality inspection result of the polyurethane sample to be inspected on the basis of the property information associated with the determined reference spectrum information.

Further, in the electronic nose device according to the second aspect of the present invention, the property information may include smell evaluation information and/or a mark indicating whether the sample is a qualified sample.

Further, the electronic nose device according to the second aspect of the present invention further may comprise a measuring unit configured to measure the polyurethane sample to be inspected so as to obtain the measured spectrum information.

Further, the electronic nose device according to the second aspect of the present invention may further comprise a spectrum information selecting unit configured to select at least one piece of reference spectrum information from the reference spectrum information of the plurality of inspected polyurethane samples; a spectrum information comparing unit configured to compare the selected at least one piece of reference spectrum information to the measured spectrum information; and an analyzing unit configured to analyze the main chemical substances that cause spectrum information differences.

Further, the electronic nose device according to the second aspect of the present invention may further comprise a spectrum information selecting unit configured to select at least one piece of reference spectrum information from the reference spectrum information of the plurality of inspected polyurethane samples; a unit for comparing specific substances, which compares the selected at least one piece of reference spectrum information to the measured spectrum information for one or more substances constituting the polyurethane sample to be inspected; and a content difference determining unit which determines a content difference of the one or more substances in at least one inspected polyurethane sample corresponding to the at least one reference spectrum information and in the polyurethane sample to be inspected corresponding to the measured spectrum information.

Further, the electronic nose device according to the second aspect of the present invention further comprises a detecting unit configured to detect release of the one or more chemical substances constituting the polyurethane sample to be inspected in the air.

Further, in the electronic nose device according to the second aspect of the present invention, the smell evaluation information may be information based on VDA270 criterion.

Further, the electronic nose device may be provided, which comprises a memory, a processor, and a computer program stored in the memory and executable on the processor, and the steps of the quality inspection method according to the first aspect of the present invention are carried out when the processor executes the computer program.

Further, in the electronic nose device according to the third aspect of the present invention, the memory may further store databases of the reference spectrum information of the plurality of inspected polyurethane samples and the property information of inspected polyurethane samples associated with each of the reference spectrum information.

Further, in the electronic nose device according to the third aspect of the present invention, the property information may include smell evaluation information and/or a mark indicating whether the sample is a qualified sample.

Further, in the electronic nose device according to the third aspect of the present invention, the smell evaluation information may be information based on VDA270 criterion.

According to a fourth aspect of the present invention, a storage medium is provided with a computer program stored thereon, wherein the program is executed by a processor to carry out the steps of the quality inspection method according to the first aspect of the present invention.

Compared to the prior art, the present invention can achieve one or more of the following advantageous effects:

1) according to the present invention, a fast quality inspection (especially inspections about the VOC and smell) can be performed on the polyurethane material immediately after it has been manufactured and before it is made into products, thus it can be determined whether the smell of the final product is from the material itself or from mutual-interference between materials or from improper storage;

2) according to the present invention, various analyses including traceability analysis can be performed effectively to meet different needs;

3) according to the present invention, the database of polyurethane samples can be further perfected so as to make subsequent inspection more accurate;

4) according to the present invention, qualities of polyurethane samples can be inspected without smelling by assessors, thus reducing harms caused to the assessors and providing more objectivity to the testing.

### BRIEF DESCRIPTION OF THE DRAWINGS

FIG. 1 is a schematic flow chart of a method S100 for inspecting the quality of a polyurethane sample according to one embodiment of the present invention;

FIG. 2 is a schematic block diagram of an electronic nose device 100 according to one embodiment of the present invention;

FIG. 3 is a schematic block diagram of an electronic nose device 200 according to another embodiment of the present invention.

### DETAILED DESCRIPTION OF THE INVENTION

The quality inspection method for polyurethane samples, electronic nose device and storage medium of the present invention will be described in further detail below with reference to the drawings. It shall be noted that the following specific embodiments are exemplary but not restrictive, and they only intend to provide a basic understanding of the present invention, but not to limit the crucial or decisive elements or define the claimed protection scope.

The present invention will now be described with reference to illustrations of the block diagrams of the method and device as well as the block diagrams and/or flow charts of the embodiments of the present invention. It shall be understood that the flow chart illustrations and/or each block of the block diagrams as well as combinations of the flow chart illustrations and/or block diagrams can be realized by computer program instructions. These computer program instructions can be provided to general-purpose computers, dedicated computers or processors of other programmable data processing devices to form machines, so that the instructions executed by the computers or processors of other programmable data processing devices create components for realizing functions/operations specified in the flow charts and/or blocks and/or one or more flow block diagrams.

These computer program instructions can be stored in a computer-readable memory, and these instructions can instruct the computer or other programmable processors to realize functions in specific ways, so that these instructions stored in the computer-readable memory form products containing instruction components for realizing functions/operations specified in one or more blocks of the flow charts and/or block diagrams.

These computer program instructions can be loaded to the computer or other programmable data processors, so that a series of operation steps are carried out on the computer or other programmable data processors to form computer-implemented processes, thus said instructions executed on the computer or other programmable data processor provide steps for realizing the functions or operations specified in one or more blocks of the flow charts and/or block diagrams. It should also be noted that in some alternative implementations, the functions/operations indicated in the blocks may not happen according to the sequence shown in the flow chart. For example, two blocks shown in sequence may actually be carried out at substantially the same time, or they may be carried out in an inverted sequence sometimes depending on the functions/operations involved.

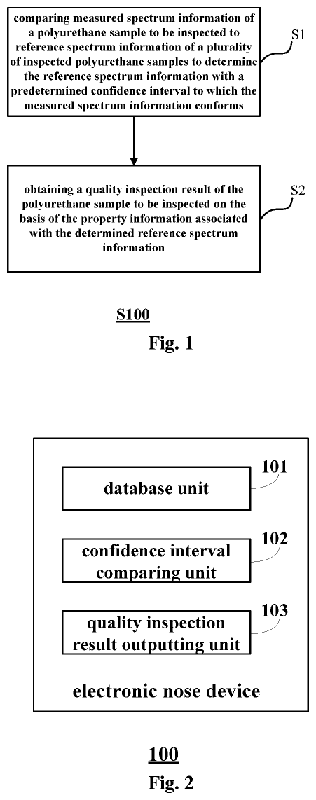

FIG. 1 is a schematic flow chart of a method S100 for inspecting the quality of a polyurethane sample according to one embodiment of the present invention.

It shall be noted that this quality inspection method for a polyurethane sample can be used in the industrial production stage, i.e. a stage after producing the polyurethane material and before making it into products like various automobile interiors, seats, dashboards, mattresses, sofa, and so on. In addition, it should be noted that the quality inspection method according to one embodiment of the present invention is not only applicable to inspection of solid state polyurethane samples but also applicable to inspection of liquid state raw materials for making the polyurethane samples, such as polyether polyol, isocyanate, etc.

As shown in FIG. 1, said method S100 comprises the following steps: comparing measured spectrum information of a polyurethane sample to be inspected to reference spectrum information of a plurality of inspected polyurethane samples to determine the reference spectrum information with a predetermined confidence interval to which the measured spectrum information conforms (step S1), wherein each of the reference spectrum information is associated with property information of corresponding inspected polyurethane samples.

In one embodiment, said step S1 can be carried out in, for example, an electronic nose device.

Further, in one embodiment, the polyurethane sample to be inspected can be measured using the electronic nose device to obtain the spectrum information thereof (i.e. the measured spectrum information). The spectrum information may be measured based on chromatographic methods such as gas chromatography or HPLC.

In addition, in one embodiment, the property information may include smell evaluation information and/or a mark indicating whether the sample is a qualified sample.

Further, in one embodiment, the smell evaluation information can be information based on VDA270 criterion, namely, it can be a score between 0-6, for example. In addition, in one embodiment, the mark indicating whether the sample is a qualified sample can be determined according to the smell evaluation information and a pre-set threshold, namely, for example, a polyurethane sample having the smell evaluation information that is scored above 3 is considered as unqualified sample, and a polyurethane sample having the smell evaluation information that is scored below 3 is considered as qualified sample.

In addition, in one embodiment, before performing the step S1, a plurality of known polyurethane samples can be measured using the electronic nose device to obtain their spectrum information (i.e. reference spectrum information), and the property information of said plurality of known polyurethane samples are input into the electronic nose device, then each of the above-mentioned reference spectrum information and the corresponding property information of the inspected polyurethane samples are stored in the electronic nose device to create a database of the polyurethane samples for use in subsequent quality inspection.

Moreover, in one embodiment, when performing the step S1, a certain confidence interval can be set as desired, and the measured spectrum information of the polyurethane sample to be inspected is compared to the reference spectrum information of each of the plurality of inspected polyurethane samples to determine the confidence interval of which inspected polyurethane sample the polyurethane sample to be inspected conforms to, i.e. to determine the reference spectrum information of which inspected polyurethane sample the measured spectrum information of the polyurethane sample to be inspected is most similar to.

In one embodiment, as shown in FIG. 1, the method S100 may further comprise a step of obtaining a quality inspection result of the polyurethane sample to be inspected on the basis of the property information associated with the determined reference spectrum information (step S2).

For example, in one embodiment, a polyurethane sample to be inspected that conforms to the confidence interval of a certain inspected polyurethane sample can be considered as having property information similar to that of said inspected polyurethane sample, so the property information of said inspected polyurethane sample can be endowed to said polyurethane sample to be inspected as its quality inspection result, or the property information of said inspected polyurethane sample can be subjected to a series of processing before being endowed to said polyurethane sample to be inspected as its quality inspection result, and so on.

In addition, in one embodiment, the measured spectrum information and property information of the polyurethane sample to be inspected can be added into the database of the inspected polyurethane samples to further perfect the database.

In addition, in one embodiment, the method S100 may further comprise the steps of selecting at least one piece of reference spectrum information from the reference spectrum information of the plurality of inspected polyurethane samples; comparing the selected at least one piece of reference spectrum information to the measured spectrum information; and analyzing the main chemical substances that cause spectrum information differences. Thus pertinent traceability analysis can be performed for improvement.

In addition, in one embodiment, the method S100 may further comprise the steps of selecting at least one piece of reference spectrum information from the reference spectrum information of the plurality of inspected polyurethane samples; comparing the selected at least one piece of reference spectrum information to the measured spectrum information for one or more substances constituting the polyurethane sample to be inspected; and determining a content difference of the one or more substances in at least one inspected polyurethane sample corresponding to the at least one reference spectrum information and in the polyurethane sample to be inspected corresponding to the measured spectrum information.

In addition, in one embodiment, the method S100 may further comprise the steps of detecting release of the one or more chemical substances constituting the polyurethane sample to be inspected in the air.

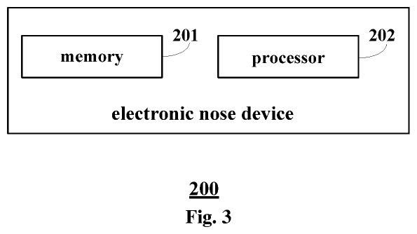

Now referring to FIG. 2, an electronic nose device 100 according to one embodiment of the present invention will be described. As shown in FIG. 2, the electronic nose device 100 comprises a database unit 101 storing databases of reference spectrum information of a plurality of inspected polyurethane samples and property information of inspected polyurethane samples associated with each of the reference spectrum information.

In one embodiment, the property information may include smell evaluation information and/or a mark indicating whether the sample is a qualified sample.

Further, in one embodiment, the smell evaluation information can be information based on VDA270 criterion, namely, it can be a score between 0-6, for example. In addition, in one embodiment, the mark indicating whether the sample is a qualified sample can be determined according to the smell evaluation information and a pre-set threshold, namely, for example, a polyurethane sample having the smell evaluation information that is scored above 3 is considered as unqualified sample, and a polyurethane sample having the smell evaluation information that is scored below 3 is considered as qualified sample.

In addition, in one embodiment, a plurality of known polyurethane samples can be measured using the electronic nose device to obtain their spectrum information (i.e. reference spectrum information), and the property information of said plurality of known polyurethane samples are input into the electronic nose device, then said reference spectrum information and the corresponding property information of the inspected polyurethane samples are stored in the electronic nose device to create a database of the polyurethane samples for use in subsequent quality inspection.

In one embodiment, as shown in FIG. 2, the device 100 may further comprise a confidence interval comparing unit 102 configured to compare the measured spectrum information of the polyurethane sample to be inspected to the reference spectrum information of a plurality of inspected polyurethane samples so as to determine the reference spectrum information with a predetermined confidence interval to which the measured spectrum information conforms.

Further, in one embodiment, the device 100 may further comprises a measuring unit (not shown in the figure) configured to measure the polyurethane sample to be inspected to obtain the measured spectrum information.

Moreover, in one embodiment, in the confidence interval comparing unit 102, a certain confidence interval can be set as desired, and the measured spectrum information of the polyurethane sample to be inspected is compared to the reference spectrum information of each of the plurality of inspected polyurethane samples to determine the confidence interval of which inspected polyurethane sample the polyurethane sample to be inspected conforms to, i.e. to determine the reference spectrum information of which inspected polyurethane sample the measured spectrum information of the polyurethane sample to be inspected is most similar to.

It shall be noted that the inspection of the polyurethane sample can be applied to the industrial production stage, i.e. a stage after producing the polyurethane material and before making it into products like various automobile interiors, seats, dashboards, mattresses, sofa, and so on. In addition, it should be noted that the present invention is not only applicable to inspection of solid state polyurethane samples but also applicable to inspection of liquid state raw materials for making the polyurethane samples, such as polyether polyol, isocyanate, etc.

In one embodiment, as shown in FIG. 2, the device 100 may further comprises a quality inspection result outputting unit 103 configured to obtain a quality inspection result of the polyurethane sample to be inspected on the basis of the property information associated with the determined reference spectrum information.

For example, in one embodiment, a polyurethane sample to be inspected that conforms to the confidence interval of a certain inspected polyurethane sample can be considered as having property information similar to that of said inspected polyurethane sample, so the property information of said inspected polyurethane sample can be endowed to said polyurethane sample to be inspected as its quality inspection result, or the property information of said inspected polyurethane sample can be subjected to a series of processing before being endowed to said polyurethane sample to be inspected as its quality inspection result, and so on.

In addition, in one embodiment, the measured spectrum information and property information of the polyurethane sample to be inspected can be added into the database of the inspected polyurethane samples to further perfect the database.

In addition, in one embodiment, the device 100 may further comprise a spectrum information selecting unit (not shown in the figure) configured to select at least one piece of reference spectrum information from the reference spectrum information of the plurality of inspected polyurethane samples.

Further, in one embodiment, the device 100 may further comprise a spectrum information comparing unit (not shown in the figure) configured to compare the selected at least one piece of reference spectrum information to the measured spectrum information.

Further, in one embodiment, the device 100 may further comprise an analyzing unit (not shown in the figure) configured to analyze the main chemical substances that cause spectrum information differences. Thus pertinent traceability analysis can be performed for improvement.

In addition, in one embodiment, the device 100 may further comprise a spectrum information selecting unit (not shown in the figure) for selecting at least one piece of reference spectrum information from the reference spectrum information of the plurality of inspected polyurethane samples.

Further, in one embodiment, the device 100 may further comprise a unit for comparing specific substances (not shown in the figure), which compares the selected at least one piece of reference spectrum information to the measured spectrum information for one or more substances constituting the polyurethane sample to be inspected.

Further, in one embodiment, the device 100 may further comprise a content difference determining unit (not shown in the figure) which determines a content difference of the one or more substances in at least one inspected polyurethane sample corresponding to the at least one reference spectrum information and in the polyurethane sample to be inspected corresponding to the measured spectrum information.

In addition, in one embodiment, the device 100 may further comprise a detecting unit (not shown in the figure) configured to detect release of the one or more chemical substances constituting the polyurethane sample to be inspected in the air.

Although the above descriptions focus on embodiments of the quality inspection method for polyurethane samples and the electronic nose device, the present invention is not limited to these embodiments. The present invention can also be implemented as a computer program for realizing functions of the electronic nose device or as a storage medium storing said computer program.

FIG. 3 is a schematic block diagram of an electronic nose device 200 according to another embodiment of the present invention. As shown in FIG. 3, the electronic nose device 200 comprises a memory 201, a processor 202 and a computer program stored in the memory 201 and executable on the processor 202, and the steps of the quality inspection method S100 according to one embodiment of the present invention are carried out when the processor executes the computer program.

In one embodiment, the memory 201 further stores databases of the reference spectrum information of the plurality of inspected polyurethane samples and the property information of inspected polyurethane samples associated with each of the reference spectrum information.

In one embodiment, the property information may include smell evaluation information and/or a mark indicating whether the sample is a qualified sample.

Further, in one embodiment, the smell evaluation information can be information based on VDA270 criterion, namely, it can be a score between 0-6, for example. In addition, in one embodiment, the mark indicating whether the sample is a qualified sample can be determined according to the smell evaluation information and a pre-set threshold, namely, for example, a polyurethane sample having the smell evaluation information that is scored above 3 is considered as unqualified sample, and a polyurethane sample having the smell evaluation information that is scored below 3 is considered as qualified sample.

Moreover, as mentioned above, the present invention can also be implemented as a storage medium storing a program executed by the processor to carry out the quality inspection method S100 according to one embodiment of the present invention.

The storage medium can be various storage mediums such as discs (e.g. magnetic disc, optical disc, etc.), cards (e.g. memory card, optical card, etc.), semiconductor memories (e.g. ROM, non-volatile memory, etc.) and tapes (e.g. magnetic tape, cassette tape, etc.).

By storing in these storage mediums the computer program that enables the computer to perform the quality inspection method for polyurethane samples as described in the above embodiments or the computer program that enables the computer to achieve the functions of the electronic nose device as described in the above embodiments and circulating such storage mediums, low cost can be realized and portability and universality can be improved.

Besides, said storage medium is loaded on the computer which reads the computer program stored in the storage medium and stores it in the memory, then the processors (Central Processing unit (CPU), Micro Processing Unit (MPU)) of the computer read said computer program from the memory and execute it, thus the quality inspection method for polyurethane samples as described in the above embodiments can be carried out and the functions of the electronic nose device as described in the above embodiments can be achieved.

Those ordinarily skilled in the art shall understand that the present invention is not limited to the above embodiments, and it can be implemented in many other forms without departing from its spirit and scope. Therefore, the exhibited examples and embodiments are illustrative rather than restrictive, and the present invention may encompass various modifications and replacements as long as they do not depart from the spirit and scope of the present invention as defined in the appended claims.

1. A quality inspection method for a polyurethane sample, the quality inspection method comprising the following steps:
comparing measured spectrum information of a polyurethane sample to be inspected to reference spectrum information of a plurality of inspected polyurethane samples to determine the reference spectrum information with a predetermined confidence interval to which the measured spectrum information conforms, wherein each of the reference spectrum information is associated with property information of corresponding inspected polyurethane samples; and
obtaining a quality inspection result of the polyurethane sample to be inspected on the basis of the property information associated with the determined reference spectrum information.

2. The quality inspection method according to claim 1, wherein the property information includes smell evaluation information and/or a mark indicating whether the sample is a qualified sample.

3. The quality inspection method according to claim 1, further comprising the step of measuring the polyurethane sample to be inspected to obtain the measured spectrum information.

4. The quality inspection method according to claim 3, further comprising the steps of
selecting at least one piece of reference spectrum information from the reference spectrum information of the plurality of inspected polyurethane samples;
comparing the selected at least one piece of reference spectrum information to the measured spectrum information; and
analyzing the main chemical substances that cause spectrum information differences.

5. The quality inspection method according to claim 3, further comprising the steps of
selecting at least one piece of reference spectrum information from the reference spectrum information of the plurality of inspected polyurethane samples;
comparing the selected at least one piece of reference spectrum information to the measured spectrum information for one or more substances comprised in, in particular constituting the polyurethane sample to be inspected; and
determining a content difference of the one or more substances in at least one inspected polyurethane sample corresponding to the at least one reference spectrum information and in the polyurethane sample to be inspected corresponding to the measured spectrum information.

6. The quality inspection method according to claim 3, further comprising the step of detecting release of the one or more chemical substances constituting the polyurethane sample to be inspected in the air.

7. The quality inspection method according to claim 2, wherein the smell evaluation information is information based on VDA270 criterion.

8. An electronic nose device, characterized by comprising:
a database unit storing databases of reference spectrum information of a plurality of inspected polyurethane samples and property information of inspected polyurethane samples associated with each of the reference spectrum information;
a confidence interval comparing unit configured to compare the measured spectrum information of the polyurethane sample to be inspected to the reference spectrum information of the plurality of inspected polyurethane samples to determine the reference spectrum information with a predetermined confidence interval to which the measured spectrum information conforms; and
a quality inspection result outputting unit configured to obtain a quality inspection result of the polyurethane sample to be inspected on the basis of the property information associated with the determined reference spectrum information.

9. The electronic nose device according to claim 8, wherein the property information includes smell evaluation information, a mark indicating whether the sample is a qualified sample, or a combination thereof.

10. The electronic nose device according to claim 8, further comprising: a measuring unit configured to measure the polyurethane sample to be inspected so as to obtain the measured spectrum information.

11. The electronic nose device according to claim 10, further comprising:
a spectrum information selecting unit configured to select at least one piece of reference spectrum information from the reference spectrum information of the plurality of inspected polyurethane samples;
a spectrum information comparing unit configured to compare the selected at least one piece of reference spectrum information to the measured spectrum information; and
an analyzing unit configured to analyze the main chemical substances that cause spectrum information differences.

12. The electronic nose device according to claim 10, further comprising:
a spectrum information selecting unit which selects at least one piece of reference spectrum information from the reference spectrum information of the plurality of inspected polyurethane samples;
a unit for comparing specific substances, which compares the selected at least one piece of reference spectrum information to the measured spectrum information for one or more substances constituting the polyurethane sample to be inspected; and
a content difference determining unit which determines a content difference of the one or more substances in at least one inspected polyurethane sample corresponding to the at least one reference spectrum information and in the polyurethane sample to be inspected corresponding to the measured spectrum information.

13. The electronic nose device according to claim 10, further comprising: a detecting unit configured to detect release of the one or more chemical substances constituting the polyurethane sample to be inspected in the air.

14. The electronic nose device according to claim 8, further comprising a memory, a processor, and a computer program stored in the memory and executable on the processor, and the computer program, when executed by the processor, configured to:
compare measured spectrum information of a polyurethane sample to be inspected to reference spectrum information of a plurality of inspected polyurethane samples to determine the reference spectrum information with a predetermined confidence interval to which the measured spectrum information conforms, wherein each of the reference spectrum information is associated with property information of corresponding inspected polyurethane samples; and
obtain a quality inspection result of the polyurethane sample to be inspected on the basis of the property information associated with the determined reference spectrum information.

15. The electronic nose device according to claim 14, wherein the memory is configured to store databases of the reference spectrum information of the plurality of inspected polyurethane samples and the property information of inspected polyurethane samples associated with each of the reference spectrum information.

16. A non-transitory storage medium on which a computer program is stored, wherein the program is configured to be executed by a processor to carry out the steps of the quality inspection method according to claim 1.
